(19) United States
(12) Patent Application Publication
(10) Pub. No.: US 2004/0229300 A1
(43) Pub. Date: Nov. 18, 2004

(54) Zinc-based screening test and kit for early diagnosis of prostate cancer

(76) Inventor: Christopher J. Frederickson, Galveston, TX (US)

Correspondence Address: Benjamin Aaron Adler, ADLER & ASSOCIATES, 8011 Candle Lane, Houston, TX, 77071, US

(21) Appl. No.: 10/829,732

(22) Filed: Apr. 22, 2004

Related U.S. Application Data
(60) Provisional application No. 60/464,510, filed on Apr. 22, 2003.

(51) Int. Cl. G01N 33/574; A61K 31/315

(52) U.S. Cl. 435/7.23; 514/494

(57) ABSTRACT

The present invention provides a method of determining if an individual is at risk for prostate cancer. The method measures and compares zinc levels in a semen sample in the potential at risk individual with normal levels. A decrease in zinc level is indicative of a risk for prostate cancer. Further provided is a zinc-based diagnostic kit for prostate cancer.

### CROSS-REFERENCE TO RELATED APPLICATIONS

This non-provisional application claims benefit of priority of provisional U.S. Ser. No. 60/464,510, filed Apr. 22, 2003, now abandoned.

### FEDERAL FUNDING LEGEND

This invention was produced in part using funds obtained through a SBIR grant 1R43CA096354-01 from the National Institutes of Health. Consequently, the federal government has certain rights in this invention.

### BACKGROUND OF THE INVENTION

1. Field of the Invention

The present invention relates generally to the field of prostate cancer diagnosis. More specifically, the present invention relates to a zinc-based screening test and kit for early diagnosis of prostate cancer.

2. Description of the Related Art

Prostate cancer kills about 40,000 men in the United States each year and there are approximately 330,000 new cases diagnosed annually. Prostate cancer is second only to lung cancer in mortality to men. Castration, treatment with anti-androgens, and prostatectomy with its associated urogenital risk, are all treatments that seriously compromise the quality of male life.

Currently, serum prostate-specific antigen (PSA), a serine protease, level and prostate digital rectal exams are the only early diagnostic tests in routine use to screen for prostate cancer. However, small, aggressive tumors can be missed by digital rectal exams and even by needle biopsy, and only modest increases in prostate-specific antigen, i.e., below the 4 ng/mL threshhold between normal and elevated PSA levels, are generated by these tumors. These aggressive tumors have the potential to suddenly dedifferentiate and grow, spread, and metastasize rapidly.

In addition to such lethal false negatives, false positives also plague the PSA test, causing unneeded tests and medical expense and distress to patients. An NCI fact sheet (1) indicates that among men above 50 years old, an age group of men most susceptible to prostate cancer, 80% of those having PSA test levels above 4 ng/mL will turn out to not have prostate cancer. The NCI Fact Sheet notes the need for a prostate cancer screen with improved ability to differentiate between prostate cancer and benign conditions such as prostatitis, benign prostatic hypertrophy (BPH) or enlarged prostate, inflammation and infection, and to differentiate between slow-growing and fast-growing cancers.

The NCI makes only a guarded recommendation for prostate cancer screening of asymptomatic men (1), and the American College of Preventive Medicine flatly recommends against it, as do some individual practitioners. Urologists generally favor screening, as does the American Cancer Society, while other groups and authors of reviews equivocate. At issue is whether the screening information leads to a clear course of action that can improve the quality or duration of life.

This controversy likely reflects the inadequacy of the diagnostic information obtained from the existing screening methods. Consider the patient who suffers a false negative, for example, in which a small tumor, e.g., T1a,b, T2a, is missed by digital rectal exams and missed in needle biopsy, even an ultrasound-guided, 6-sector biopsy, and does not raise the serum PSA to alarming levels, i.e. PSA below 4. Depending on the grade of tumor, a patient with a Gleason Pattern GP 4-5 tumor could have a metastatic disease with poor prognosis within a year whereas a patient with a GP 1-2 tumor might experience little changes in a year. Since most prostate cancers are slow-growing, there is a clear need for a routine diagnostic screen that can pick up prostate cancer before it is large enough to produce symptoms.

Zinc is the most ubiquitous heavy metal in the human body. In the male reproductive system semen has 3 mM zinc, which is approximately 1000-fold more than those found in saliva, tears, vaginal secretions, urine or blood. Indeed, ejaculate contains so much zinc that a zinc-sensitive dye is used routinely by police to find semen at crime scenes. Why zinc is present in semen has not been established clearly. Some researchers speculated that the zinc is an antimicrobial for cleansing the urethra. It is also true that zinc suppresses the proteolytic activity of PSA, the enzyme that cleaves seminal globular proteins to “liberate” spermatozoa, suggesting a possible zinc-mediated control process of spematozoan mobility. A role for zinc in citrate metabolism has also been noted. Finally, spermatozoa are richly endowed with zinc both in their cytosol and on their exterior, suggesting that seminal fluid might be needed to maintain a spermatozoan zinc pool.

Regardless of the function of zinc in semen, the source of zinc appears to be in part from the testes, which concentrates zinc in and on the spermatozoa, and in part from the secretory cells lining the ducts of the lateral lobes of the prostate gland. At the fine and ultrastructural level, the zinc in the prostate tubules is concentrated at the apical ends of the secretory cells, in the interstities between the cells, and most massively, in the lumen of the seminal ducts.

Physiologically, the epithelial secretory cells show relatively high velocity uptake of zinc that is driven by testosterone. Thus, one assumes that the epithelial secretory cells take up zinc, sequester it in secretory granules, and secrete the contents of the granules into the lumen, thereby generating the high zinc content of the semen (FIG. 1). The immunostaining methods developed for the zinc transporters ZnT-1, ZnT-2, and ZnT-3 fail to label the prostate epithelial cells, and thus the zinc influxing transporter has not yet been identified, although a ZIP protein has been suggested as important to this transport.

There is an overwhelming, in fact, almost unanimous, consensus from many laboratories worldwide that the prostate gland has a uniquely high zinc content which is localized to the lateral lobes and that the prostate loses from 50% to 90% of that zinc in prostate cancer. In contrast, the zinc levels increase in benign prostatic hypertrophy (BPH) and show no consistent change in prostatitis. Perhaps the best reference on the changes in zinc in the prostate in cancer and BPH is the analysis published by Zaichicks et al. who compiled data from 16 prior studies as well as their own (2).

Only one of the 17 papers reviewed failed to find decreased zinc in prostate cancer, and the other 16 all showed declines in cancer, with 15 of 16 showing ratios of diseased/control within the fairly narrow range of 0.15 and 0.55 (2). On average across the 17 studies, the zinc level was found to double in BPH with mean and median ratios 2.25 and 1.98, respectively. Other papers not covered in the Zaichick's review have also found the same basic pattern of prostate zinc changes in cancer and BPH (3-4).

Since most of the zinc in the prostate is concentrated in the lumen and secretory surfaces of the seminal tubules, e.g. in the secretory fluids, the observed drop of 50-90% in total zinc content would be expected to require a significant drop in the zinc content of the seminal fluid. This is confimed by empirical data obtained both from patients with stage T3-T4 tumors which showed a 95% decrease in zinc in ejaculate (2), and from patients with palpable tumors which showed an 84% decrease in zinc in post-prostatic massage fluid (5). In benign prostatic hypertrophy, the zinc level was found to be either unchanged (2) or increased (5).

In contrast to the consensus findings on significant changes in zinc in prostate tissue and secretions, the literature on zinc in blood serum in prostate cancer varies between a slight decrease (6), an increase in a rat model (7) and no change (8). While disappointing from the clinical-diagnostic perspective, this is not surprising biologically. Indeed, it would be surprising if the zinc metabolism of the prostate alone could alter total body burden or serum buffering of zinc. Hence, the consensus is that zinc in blood is not a viable marker for prostate cancer.

The “ideal” prostate screening test should reliably detect even the small, nonpalpable tumors, e.g., T1a-c, T2a, that generate only modest increases in serum PSA, i.e., below 4 ng/mL, but have the potential to dedifferentiate rapidly to Gleason pattern 4-5 and thus grow and metastasize rapidly. There is a realistic chance that semen zinc measures may be a key to such an “ideal” diagnostic. After all, it is plausible that one of the first steps in prostate epithelial cell dedifferentiation would be to turn off the molecular machinery of zinc influxing. Some indirect evidence suggest this is the case (3). This would mean that semen zinc levels might be a sensitive and selective cancer indicator.

Unfortunately, measuring zinc in complex biological matrices such as semen and determining the sizes of the different “pools” of zinc and the changes, if any, in these multiple zinc pools is a daunting bioanalytic problem. Thus, the literature on zinc and prostate cancer is alarmingly error ridden. For example, estimates in the scientific literature of total zinc in prostate tissue and total zinc in semen vary over an absolute range of nearly 100-fold (2).

The inventor has recognized a need in the art for improvements in the development and uses of state of the art bioanalytical methods to measure the distribution, speciation and concentrations of zinc in prostate tissue and seminal fluid. Specifically, the prior art is deficient in a zinc-based diagnostic test for prostate cancer. The present invention fulfills this long-standing need and desire in the art.

### SUMMARY OF THE INVENTION

The present invention is directed to a method of screening an individual at risk for prostate cancer. The method comprises collecting a semen sample from the individual and measuring the level of zinc in the sample. The zinc level from the at risk individual is compared with zinc levels found in a normal individual where a decreased zinc level in the individual compared to the normal level indicates a risk of developing prostate cancer, thereby screening the individual.

The present invention also is directed to a related method of screening an individual at risk for prostate cancer. Further to the method described supra, the level of prostate specific antigen is measured in the potential at risk individual. The PSA level in the individual is compared to PSA levels in normal individuals where an increased PSA level compared to normal further indicates a risk for prostate cancer.

The present invention is directed further to a kit for early diagnosis of prostate cancer in an individual. The kit comprises a cassette containing a fluorescent probe contained there in and a specimen container for a semen sample comprising a means permeable to free zinc ions but impermeable to the fluorescent probe. The specimen container is insertable into the cassette. The kit also has a fluorescence reader that comprises a means to convert fluorescence values of a fluorescent probe-zinc complex to zinc level values. The kit further comprises a chart to convert the zinc level values to a status of normal prostate normal, pre-disposition to prostate cancer or prostate cancer in the individual.

The present invention also is directed to a related kit for early diagnosis of prostate cancer. The cassette contains apoCA and a fluorescence reporter and the specimen container comprises a membrane permeable to free zinc but is not permeable to the apoCA nor the fluorescent reporter. The fluorescence reader and the chart are as described above.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention as well as others which will become clear are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIG. 1 shows the proposed life cycle of zinc in prostate epithelial cells. Indirect evidence suggests that zinc pump expression may be down regulated early in cancer, thus causing reduced zinc in semen.

FIG. 2 shows four methods of staining and measuring free zinc in or on spermatozoa. Znpyr and TSQ are fluorimetric, whereas AMG gives high resolution even in the EM. Scale bar is ˜2 mm for the light micrographs.

FIG. 3 is a gel depicting the mix of proteins in seminal plasma. The gel was run at pH 4-7 with molecular weight markers of 250, 150, 100, 75, 50, 37, 25, 20, 15, and 10 KDas.

FIG. 4A shows zinc staining (brown-black) in the lateral lobes of the prostate in rat.

FIG. 4B shows that zinc staining (black) fills the lumen of the lateral prostate tubule of the rat in these plastic-embedded sections. Note that the epithelial cells have only sparse grains in their apical (secretory) ends, as seen in the electron micrograph (lower panel).

FIG. 5 shows accurate zinc measurements in the left and right brain structures of 6 rats. Note the close agreement of measurements. Median L-R error is about 2% for brain regions with total zinc burdens of about 800 ng.

FIG. 6A shows zinc measurements in a genuine biological matrix (ACSF). The results show good stability and sensitivity. Note clear detection of 45 nM, which corresponds to 292 pg of total zinc.

FIG. 6B shows the apoCA zinc sensing method gives a robust, ratiometric shift in fluorescence anisotropy with Zn levels in the sub-picomolar levels.

FIG. 6C shows two different mutants of carbonic anhydrase having different on-rates (and have different affinities) for zinc. The fluorescence indicates zinc binding by ABDN.

FIG. 7 shows tissue distribution of total zinc (false color image) by synchrotron-induced x-ray fluorescence. This may be compared and contrasted with the image of free zinc (right panel). Both are important for understanding zinc in the prostate. Images are from rat brain; about 2 mm×2 mm.

FIG. 8 shows zinc staining of globular proteins in ejaculate. Blue fluorescence of zinc (TSQ) can be quantified whereas the black silver grains (AMG) give a higher resolution localization in the EM.

### DETAILED DESCRIPTION OF THE INVENTION

In one embodiment of the present invention there is provided a method of screening an individual at risk for prostate cancer, comprising collecting a semen sample from the individual; measuring the level of zinc in the sample; comparing the zinc level from the at risk individual with zinc levels found in a normal individual; and correlating a decreased zinc level in the individual compared to the normal level to a risk of developing prostate cancer, thereby screening the individual. Further to this embodiment the method comprises measuring a level of prostate specific antigen in the at risk individual; comparing the PSA level from the at risk individual with PSA levels found in a normal individual; and correlating an increased PSA level in the individual compared to the normal level to a risk of developing prostate cancer.

In all aspects of these related embodiments the zinc may be free zinc, microligand-bound zinc, protein-bound zinc or zinc carried by spermatozoa. In one aspect of this embodiment the semen sample may be whole seminal fluid or seminal plasma. In this aspect the zinc comprising the semen sample is measured by stable isotope dilution mass spectrometry or fluorescent spectrometry. An example of fluorescent spectrometry is a ratiometric fluorescence method. The ratiometric fluorescent method may employ an apoCA and a fluorescent reporter. Examples of a fluorescent reporter are dansylamide and ABDN. The fluorescent spectrometry may employ a fluorescent probe, The fluorescent probes may be a Zinpyr, a zin-napthopyr, such as ZNP-1, TSQ, Fluo-zinc, or coumazin. Examples of Zinpyr may be ZP-1, ZP-4 or ZP-8.

In another aspect of this embodiment the semen sample may be spermatozoa, cytosol of spermatozoa or seminal globulin protein. In this aspect the zinc comprising the semen sample may be measured by microspectrofluorimetric methods or silver staining autometalography. Microspectrofluorimetric methods may employ TSQ or cell-impermeable stains to stain the zinc comprising the semen sample. Cell-impermeable stains may be Newport Green, a Zinpyr or a Zin-napthopyr. Examples of a Zinpyr stain are ZP-1, ZP-4 and ZP-8. An example of Zin-napthopyr is ZNP-1.

In still another embodiment of the present there is provided a kit for early diagnosis of prostate cancer in an individual, comprising a cassette containing a fluorescent probe; a specimen container for a semen sample comprising a means permeable to free zinc ions but impermeable to said fluorescent probe, said specimen container insertable into said cassette; a fluorescence reader comprising means to convert fluorescence values of a fluorescent probe-zinc complex to zinc level values; and a chart to convert said zinc level values to a status of normal prostate, pre-disposition to prostate cancer or prostate cancer in the individual.

Further to this embodiment the kit may comprise a detergent. An example of a detergent is Triton-X 100. Additionally, the kit may comprise an antibody immobilized on a substrate where the antibody is directed against a zinc-binding ligand in semen.

In all aspects of this embodiment the fluorescent probe may be apoCA and a fluorescent reporter, Zinpyr, a zin-napthopyr, TSQ, Fluo-zinc, or coumazin. Examples of a fluorescent reporter are dansylamide and ABDN. Representative Zinpyrs are ZP-1, ZP-4 and ZP-8. The zin-napthopyr may be ZPN-1. The means permeable to free zinc ions but not to said fluorescent probe may be a dialysis membrane or a molecular sieve. The semen sample may be whole seminal fluid.

In a related embodiment of the present invention there is provided a kit for early diagnosis of prostate cancer in an individual, comprising a cassette having an apoCA and a fluorescence reporter contained therein; a specimen container for a semen sample comprising a membrane permeable to free zinc ions but impermeable to the apoCA and the fluorescent reporter such that the specimen container is insertable into the cassette; a fluorescence reader comprising means to convert fluorescence values of a holoCA-fluorescence reporter complex to zinc level values; and a chart to convert the zinc level values to a status of normal prostate, pre-disposition to prostate cancer or prostate cancer in the individual.

In this related embodiment the kit further may comprise the detergent and antibodies as described supra. In all aspects of this embodiment the specimen container may be a pouch comprising a dialysis membrane. The fluorescent reporter may be dansylamide or ABDN. The semen sample may be whole seminal fluid.

The present invention provides analytical tools for measuring the amount and speciation of zinc in semen. These tools facilitate basic research and may result in a zinc-based diagnostic kit for prostate cancer. It is contemplated that a combined measurement of semen zinc levels, which fall in cancer, and serum PSA levels, which rise in cancer, could provide sensitive and selective early diagnosis for prostate cancer. Test kits for routine testing of seminal zinc in the clinic or even at home can be developed.

A valid zinc diagnostic test for prostate cancer requires speciation of semen zinc in that the fall in semen zinc at the onset of prostate cancer is not equally specific to the different semen zinc pools, i.e. free zinc, microligand bound zinc, small protein bound zinc, large protein bound zinc, and spermatozoan zinc. Identification of the semen zinc pool(s) which change earliest and most consistently at the onset of prostate cancer is necessary to optimize the sensitivity and selectivity of the prostate screening test.

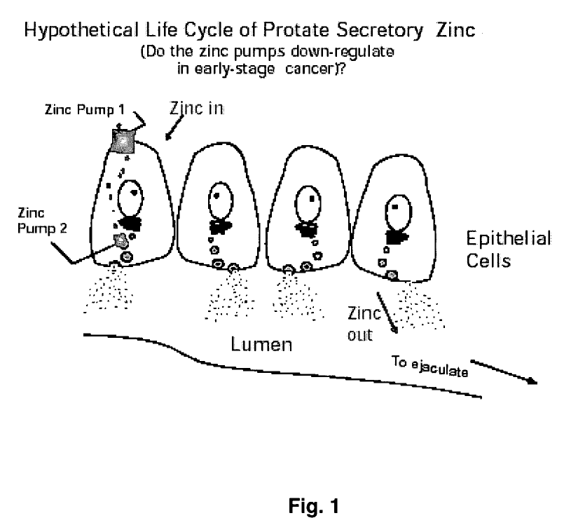

For the present invention, a key observation is that zinc content of semen and prostate tissue apparently drops rapidly in the earliest stages of prostate cancer (2). This fall in zinc may be due to down-regulation of the zinc influxing pumps of prostate cells in the earliest stages of endothelial cell dedifferentiation and proliferation (FIG. 1). Whatever the mechanism might be, the diagnostic value of this metabolic change could be life-saving.

For a zinc-based cancer diagnostic, one must determine exactly how much zinc is in the ejaculate, how it is distributed among which groups of endogenous ligands, what the within and between individual variability is and, of course, which of the zinc pools will offer the best early diagnostic for cancer. Thus, the crucial steps in the development of zinc-based prostate cancer screening method and diagnostic kit include elucidating exactly how much zinc is really in ejaculate, how zinc is distributed among the many quantitatively-important pools, such as, but not limited to, free zinc, microligand-bound zinc, small protein bound zinc, and large protein bound, and which of those pools changes earliest and most consistently at cancer onset.

The present invention develops and provides state of the art procedures for measuring the distribution, speciation and concentrations of zinc in prostate tissue and seminal fluid, i.e., ejaculate. Measures to be determined include: free versus bound zinc in seminal plasma; ligand binding, i.e., speciation, of zinc in semen; free versus bound zinc in prostate tissue; zinc concentrations in individual spermatozoa; and histochemical localization(s) of the free stainable zinc pool using Timm-Danscher fluorescence and Synchrotron X-ray fluorescence.

Using the methods developed herein, the means, ranges, and variances of zinc contents in prostate tissue and ejaculate can be determined in men with or without, as a control, prostate cancer. Methodology was developed to allow determination of: 1) free and total zinc in whole seminal fluid or ejaculate; 2) free and total zinc in seminal plasma; 3) zinc bound to specific subsets of seminal proteins; and 4) zinc concentration in individual spermatozoa.

Provided herein is a method of screening for prostate cancer by measuring the amount of zinc in semen samples. Decreased levels of zinc compared to those found in normal individual would indicate such individual is at risk of developing prostate cancer. The semen samples can be whole seminal fluid, seminal plasma, spermatozoa, cytosol of spermatozoa or seminal globulin protein.

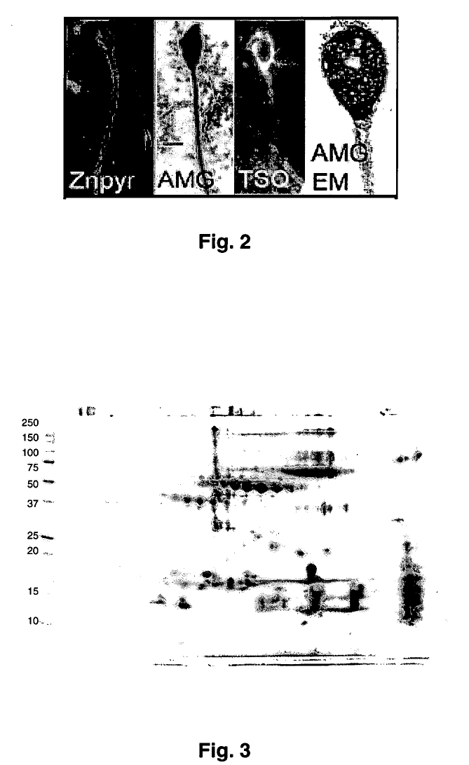

Specifically, there are three measures of zinc in semen with diagnostic potential: (i) the concentration of “free” or “rapidly-exchangeable” zinc in the semen, (ii) the concentration of zinc bound to organic ligands in the semen, such as proteins, peptides, amino acids, small molecules, and (iii) the zinc in cells, such as spermatozoa or endothelial cells that have sloughed into the semen. FIG. 2 summarizes the three main methods of localizing and/or quantitating weakly-bound zinc. These histochemical methods have different strengths and different uses.

Generally, the fluorescent methods are best for quantitation as they are stoichiometric and with the apoCA versions ratiometric. Thus, for the purpose of measuring the weakly bound zinc, the present invention uses fluorescence analysis. Among the fluorescence methods, there are further choices based on the subcellular location of the zinc to be measured. For example, the membrane impermeable apoCA will not label zinc in vesicles, nor will the “trappable” Newport green, which is metabolized in cytosol, label zinc in the cytosol. In contrast, the lipophilic stains TSQ and Zinpyr will stain zinc in intracellular organelles, cytosol, and in extracellular fluid.

Generally, free and total zinc in solution can be measured by apoCA fluorimetric method and stable isotope dilution mass spectrometry, respectively. Alternatively, microspectro-fluorimetric methods or silver staining autometalography can be used to measured zinc that is not in solution. Thus, extracellular zinc, such as zinc on the outer surfaces of spermatozoa or zinc loosely coordinated with globular proteins, can be stained with cell-impermeable stains such as Newport Green, and the fluorescein-based metal sensors Zinpyr or Zin-naphthopyr (ZNP) (9), or by TSQ. U.S. Patent Application No. 20020106697 discloses ZP-4 and ZP-8 as examples of Zinpyrs.

Moreover, the instant zinc-based screening method can be combined with PSA assays currently in use to obtain screening with enhanced accuracy. Results of decreased levels of zinc combined with increased levels of PSA compared to those found in normal individual would render prostate cancer screening more sensitive and accurate. This provides corroboration of results with a higher level of control for the screening method

A simple, inexpensive test kit with calorimetric, or even, given the $5 LED's and CCD'S on the market, a ratiometric fluorimetric measurement system is contemplated. The test may be performed in a clinic for measuring the clinically-appropriate “pool” of semen zinc. Additionally, the kit also may be a home-use test kit that would measure the amount of zinc in whole seminal fluid. Together with the information from serum PSA, for which home-use test kits are already on the market, the information from the semen zinc test could give men a new degree of certainty about the health of their prostate glands.

The kit measures zinc in one or more pools of free zinc, bound zinc or zinc in cells, as described above. Diagnosis may be based on the relative abundance of zinc in these pools and depends upon which of these pools sizes or ratios of zinc abundance in different pools is the most accurate predictor of nascent prostate cancer.

The method of measuring free zinc is to separate the free zinc from the whole semen by dialysis. Dialysis membranes with pore size of 100 MW have been shown to allow zinc to diffuse from biological fluids, while keeping fluorescent probes for zinc restrained. In the kit for free zinc, a fluorescent probe for zinc is placed on one side of a dialysis membrane or molecular sieve and the semen is placed on the other and time is allowed for the zinc to diffuse through and bind to the fluorescent probe. In addition to the dialysis step, treatment of the sample with a detergent, e.g. triton-X 100, to lyse the membranes of prostasomes can also be employed. This is because some amount of the zinc secreted by prostate epithelial cells into the semen may be sequestered in secretory prostasomes.

Many probes are known in the art. For example, a probe may be, inter alia, apoCA+a reporter, such as dansylamide or ABDN, a Zinpyr dye or stain, such as ZP-1, ZP-4 or ZP-8, a zin-napthopyr, such as ZNP-1, TSQ, Fluo-zinc, or coumazin. Others of such probes are known and readily available and can be used for this measurement.

To measure the bound zinc, additional steps of sample preparation are required. First, the zinc-binding ligands must be separated to isolate one or more of the ligands with the to-be-measured zinc. A sample of the mix of proteins that is in seminal plasma is shown in FIG. 3. To separate out from this mix of proteins, or other zinc-binding organic molecules, standard separation methods familiar to those skilled in the art are used. Such methods may be chromatography, gel separation and antibody-based extraction/purification. Not all zinc-binding ligands need to be identified or purified.

A simple immobilized antibody or aptamer can be used to trap the zinc-binding ligand of interest on a substrate, with simple washing used to remove the non-selected molecules and vehicle from the substrate. To measure the concentration of zinc in the isolated zinc-binding ligands, the zinc is first released by treating captured material with an agent such as nitric oxide, hydrogen peroxide or weak acid, or other chemical treatment methods known to those skilled in the art to release the zinc from organic ligands, to denature the zinc-binding motif thus causing the zinc to be released into the surrounding fluid. The free zinc can then be determined by the same fluorimetric methods described above. Thus the kit may further contain an antibody immobilized upon an appropriate substrate to separate zinc-binding ligands.

To measure the total zinc in cells, the cells are separated from the seminal plasma, e.g. by simple filtering. The separated cells are both lysed by triton X, as described, and bound zinc is released by the method described above. The resulting free zinc is measured by the fluorimetric methods described above.

In kit form, all of the steps above can be accomplished on simple, take-home formats, such as those utilized for measuring various analytes, e.g., glucose, cholesterol, or drugs of abuse, in bodily fluid, such as serum or urine, at home. Antibody separation is used in kits like home pregnancy tests, calorimetric tests are used in glucose, cholesterol, ketone, and other home-tests, and filtration of material including cells and cell debris out of fluid is routine in home-tests systems, e.g. in glucose test kits.

One example of such a kit comprises a “ZnDectec” cassette, a pouch comprising a dialysis bag, a small digital reader and a chart. The “ZnDectec” cassette may be a 4-5 cm container comprising a mixture of carbonic anhydrase (apoCA) and a reporter molecule, such as dansylamide (DNSA), as described. In using this kit whole seminal fluid is placed into the pouch which is designed to fit into the cassette. The free zinc ions in the sample pouch will move freely out of the pouch and into the detection cassette where the zinc ions will bind strongly to the apoCA and form the holoCA-dansylamide complex.

The pouch which is substantially depleted of free zinc ions is then removed from the cassette. The cassette is placed into a simple fluorescence reader having excitation and emission filters set to collect the fluorescence of the holoCA-dansylamide complex, but not that of DNSA or apoCA-DNSA. The fluorescence reader will convert the fluorescence values to values of zinc levels. An individual can check the chart included in the kit against the values of zinc levels obtained and determine whether the measured zinc levels fall into one of three ranges: normal, pre-disposition to prostate cancer and prostate cancer.

Beyond semen testing, zinc changes may be used as a basis for differential imaging of healthy versus cancerous prostate tissue. There are many non-toxic or benign zinc binding compounds, including such citrate, histidine, diethyldithiocarbamate, such as used in Antabuse, and clioquinol which is a USP antimicrobial, that can be taken orally and reach the prostate tissue. To image zinc, a molecule or agent which undergoes a distinctive shift in a parameter like infrared light absorption or NMR resonance frequency upon binding zinc is required. Such a zinc contrast agent would allow imaging of the healthy prostate by optoacoustic imaging or MRI. NMR contrast agents for zinc have already been demonstrated (10). Imaging of the prostate by ⁶⁹Zn or ⁷²Zn ultra-short lived nuclides has also been suggested and could be made to work with contemporary instruments (11).

The following examples are given for the purpose of illustrating various embodiments of the invention and are not meant to limit the present invention in any fashion.

### EXAMPLE 1

Subcellular and Ultrastructural Localization of Zinc

Though it is not a quantitative method, the silver AMG methods of Danscher (12) are the definitive method for determining the fine and ultrastructural localization of free or weakly-bound zinc pools. In skilled hands, these methods show as few as 10 atoms of zinc (13).

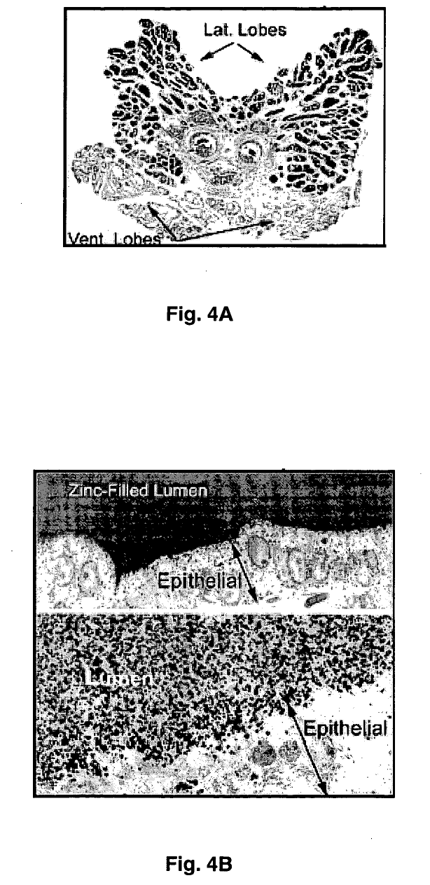

In the male reproductive system, it has been shown that zinc is gradually added to the spermatazoa as they mature through the epididymis and remains hugely enriched through the spermatazoan trip into ejaculate. FIGS. 4A and 4B show enormous amounts of zinc in the prostatic secretions within the tubules. Further, the electron microscopic view shows that the zinc is selectively concentrated in apparent secretory packets in the epithelial cells, poised, as it were, to be secreted into the zinc-rich lumen.

### EXAMPLE 2

Bulk Analysis of Total Zinc by Stable Isotope Dilution Mass Spectrometry (SIDMS)

The stable isotope dilution mass spectrometry method is a reference analytical method used to certify other laboratory methods. Manton and Frederickson developed SIDMS micro-methods for analysis of total zinc in brain tissue and Manton and Cook did the same for lead in tissue. In both cases it was found that the true amount of metal was as much as 50-fold less than that indicated by the prevailing published literature. The reason for the errors was the same in both cases: inaccurate instruments, dirty chemistry and sample preparation.

SIDMS is highly accurate in that it is a ratiometric method in which a reference amount of a stable zinc isotope, such as Zn⁶⁴, the “spike”, is added to the sample at an early stage of sample collection, and the final amount of endogenous zinc is based on the ratio of the spiked zinc isotope to the endogenous isotope at the end of sample processing. The SIDMS is routinely precise to 4 significant figures and because it does use an internal standard, the precision translates directly to absolute accuracy. Samples are subjected to acid dissolution procedures to reduce the zinc species to elemental zinc during sample processing in this method. The limit of detection for zinc is about 5 ng, so an absolute machine accuracy of approximately 5+/−0.005 ng can be obtained on any individual determination.

The accuracy of the final measure of tissue or fluid zinc concentration, however, does not depend on the instrument accuracy, but upon the degree of contamination or loss of zinc during sample preparation. This “blank” amount of zinc in the present SIDMS micromethods has been lowered to a SD of +/−0.9 ng for n=5 (14-15). Thus, in order to obtain a coefficient of variance of 5%, a reasonable standard, a minimum of 18 ng of zinc per sample is required. Given that all soft tissue has at least 60 ppm (dry) of zinc, this means no more than about 300 ug of tissue needed to be analyzed for 5% coefficient of variance.

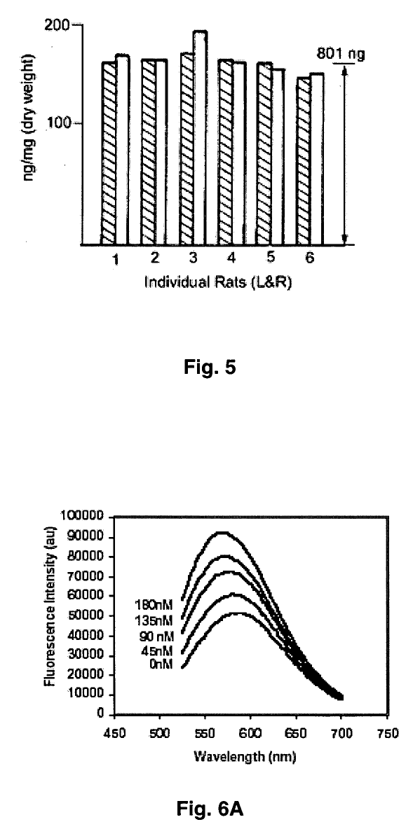

FIG. 5 depicts an example of this kind of accuracy. The left and right brain regions from individual rats were shown to vary by no more that a few percent, or in absolute terms by no more that a few nanograms.

### EXAMPLE 3

Bulk Analysis of Free Zinc by Fluorescence Ratiometric Methods

Thompson and Frederickson have developed quantitative, ratiometric fluorescence methods for determinations of “free” zinc in solutions. “Free” is of course an oversimplification. “Free” is a short-hand for reference to Zn²⁺ that is either in solution as aquozinc or is weakly coordinated to ligand(s) that have low enough affinity and fast enough off-rates to donate Zn²⁺ to the indicator probe under the conditions of the determination. Zinc classically-bound to metalloenzymes with K_{D}>>10¹² typically release little or no zinc in physiologic solutions (16-17).

The present invention employs carbonic anhydrase (CA) as the zinc detector and either ABDN or dansylamide as the fluorescent reporter for high-accuracy measurement. In operation, the fluorescent reporter binds to the CA if and only if the CA has a zinc in the “pocket”, i.e., holoCA. Upon binding to the holoCA, the reporter undergoes an increase in intensity and blue-shift in wavelength of the emission (FIG. 6A), as well as a change in fluorescence anisotropy (FIG. 6B). By starting with the apoCA, one then adds a test solution, and monitors the fraction of the reporter that is blue-shifted, or anisotropy-shifted, by the occurrence of zinc binding to the apoCA (FIG. 6A). The wavelength and anisotropy ratio measurements can be done in test tube or by confocal microscope.

An entire family of genetically-engineered CA proteins with different affinities for zinc can be generated (FIG. 6C). By simply performing a competition assay with these different CA mutants, the binding strength of zinc to different ligands in ejaculate can be measured.

### EXAMPLE 4

Distribution of Total Zinc in Tissue by X-Ray Fluorescence

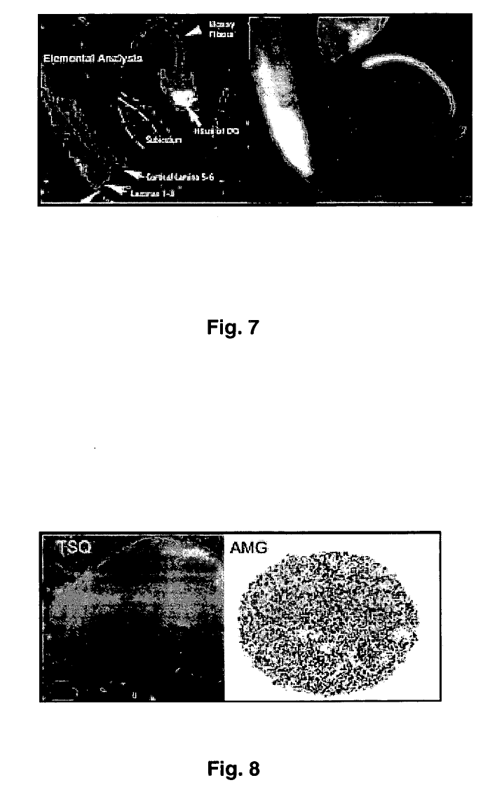

To determine the distribution of zinc among different cells, globular proteins or different regions of tissue, zinc imaging with synchrotron-induced X-ray fluorescence is used (FIG. 7). This technique can be used to determine the distribution of zinc in the different regions of the prostate gland and in different components, such as globular proteins and spermatozoa, of dried whole ejaculate as described below.

### EXAMPLE 5

Method for Fractionation of Semen Components

All containers, reagents and materials were cleaned of zinc by ion exchange, soaking in hot EDTA which chelates Zn²⁺ or hot acid, multiple rinses in 18 Mohm water, as appropriate. The success of all cleaning methods was verified by testing each procedure for the “blank” zinc contaminant level. Surfaces, e.g. soft glass, which are known to adsorb or release large amounts of zinc from solution are avoided.

Fresh ejaculate collected in tubes certified to neither adsorb zinc from samples nor to contaminate them within the limits of detection, i.e., femtogram, 10⁻¹⁵ g, was incubated at room temperature for 20 minutes to allow liquefaction. The samples were then diluted with one volume of 200 mM sucrose, 2.4 mM MgCl₂ and centrifuged at 400×g to remove intact sperm cells (18). The supernatant was stored at −80° C. for subsequent analysis, with freeze-thaw damage to proteins minimized.

Seminal plasma proteins were separated by size exclusion chromatography (19) run at 4° C. The seminal plasma samples were diluted to a protein concentration of 1 mg/mL in 150 mM NaCl, 100 mM sodium phosphate buffer (pH 7.1, buffer A) and up to 5 mL was then filtered through a 0.45 um low protein-binding filter. The diluted seminal plasma samples (2-3 mL) were then applied to a 30 cm Sephacryl S300 HR column having a resolution range of 10 to 1500 kDa (Amersham Pharmacia Biotech). The mobile phase was buffer A, delivered at a flow rate of 1 mL/min via a peristaltic pump (Gilson) and 1 mL fractions were collected. Total protein in the eluted fractions was determined spectrophotometrically by 214 nm absorbance.

Total zinc content, i.e., free plus bound, of each semen component fraction was then determined by stable isotope dilution mass spectrometry and free zinc was determined by the recently developed apoCA fluorimetric method (16). The latter method for free zinc is a fluorescence ratiometric method in which a fluorescent reporter molecule such as ABDN binds to a zinc sensor molecule, the metalloenzyme carbonic anhydrase, CA, if and only if the CA has a zinc in the “pocket.” The zinc-containing holoenzyme increases the fluorescence of the reporter.

ApoCA was prepared by removing the Zn²⁺ with dipicolinate and dialysis against a zinc chelator. The apoCA was then mixed with the fluorescent reporter, both at 2 mM, in 50 mM HEPES-buffer. When there is no Zn²⁺ in the fraction, i.e., less than the femtogram detection limit, the apoCA remains without zinc and does not bind to the fluorescent reporter, which emits its native fluorescence. When Zn²⁺ is present in the fraction, it binds stoichiometrically to the CA (K_{D} of 4 pM).

The resulting holoCA binds to the reporter, causing a shift in its emission wavelength from 600 nm to 560 nm and an 8-fold increase in emission intensity. This system readily measures zinc in fluids from pM levels up. For Zn²⁺ levels well above the K_{D}, for example, low μM levels, the percent-occupancy approach is used in which the upper limit of the fluorescence sensitivity is set by the concentration of apoCA used and the lower limit is about 1% of that. For example, with 100 μM of apoCA and 100 μM of ABD-N, the fluorescence shift will be maximal at 100 μM Zn²⁺ and is just detectable at about 0.1 to 1.0 μM.

The chromatography column is calibrated regularly with molecular weight standards (Sigma) and a parallel, calibrated column is used to resolve zinc-containing CA II (Sigma) to demonstrate efficacy of fractional zinc determination. Because carbonic anhydrase is the basis for the free zinc assay, the use of carbonic anhydrase holoenzyme with zinc and carbonic anhydrase apoenzyme with zinc removed provides an internal reference for total zinc as a fraction of total protein.

### EXAMPLE 6

Measurement of Zinc in Fluids

To measure zinc in a particular fluid, such as the whole semen plasma, one starts with an apoCA-ABDN solution at 10 times the expected zinc concentration. An aliquot of plasma is added and a fluorescence spectrum is obtained. The magnitude of the emission peak shift relative to a control sample is observed. By appropriate dilution of the unknown, one then brings the sample into the right zinc concentration range for the final spectrum.

Calibration curves are run by the method of standard additions, using the matrix, e.g. seminal plasma, as the vehicle and adding zinc. Zinc chelators such as CaEDTA are used to quench the fluorescence in order to verify that the emission shift is indeed due to zinc. SIDMS verifies the final concentration of zinc bound to the carbonic anhydrase after the carbonic anhydrase is isolated by dialysis, providing a final verification of the absolute accuracy of the method.

Access to an entire family of genetically engineered carbonic anhydrase proteins having a range of affinities for zinc would allow measurement of the binding strength of zinc to different ligands in ejaculate by simply competing for the zinc with the different carbonic anhydrase mutants (20-21).

### EXAMPLE 7

Measurement of Zinc That is not in Solution

To measure free zinc in material that is not in solution, such as in the cytosol of individual spermatozoa or in seminal globular proteins, microspectrofluorimetric methods developed by the inventor for measuring zinc in brain tissue were used (16,22). In this method, the material is stained to show the zinc pool of interest. Extracellular zinc, such as zinc on the outer surfaces of spermatozoa or zinc loosely coordinated with globular proteins, can be stained with either cell-impermeable Newport Green, or by TSQ (FIG. 8). Each stain has its particular strengths and weaknesses in this application. The material is stained, smeared on slides and the fluorescence is quantified in a fluorescence microscope and quantitative images captured on a laser-scanned confocal instrument and a cooled CCD camera.

The distribution of total zinc in the different regions of the prostate gland and in different components, e.g., globular proteins and spermatozoa, of dried whole ejaculate can be determined by Synchrotron-induced X-ray fluorescence of zinc (23). The distribution of free zinc can be determined by a series of histoanalytical methods specific to the subcellular localization of the zinc (12).

### EXAMPLE 8

Clinical Studies on Zinc-Based Early Detection of Prostate Cancer

A large-scale, longitudinal, prospective study will focus on men who are at-risk for prostate cancer, but have no clinical signs or serum indicators. The key question is whether over a 3-4 year span one can see in men who develop cancer that earlier changes in prostatic zinc secretions were an advance warning sign. Most plausibly, one might expect that the best warning would turn out to be a change in one specific pool of semen zinc with some correlated change in the blood serum PSA, or the serum free PSA/bound PSA ratio. Such a set of interrelated changes might be expected if, for example, dedifferentiating epithelial cells cease sequestering zinc in the seminal fluid and simultaneously clog seminal ducts, causing PSA to leak into blood serum.

General Subject Recruiting and Screening

Telephone screening of volunteers will be done to find 10 who meet general criteria and each prospective subject will be sent to a local health-care clinic and tested for HIV, hepatitis, TB, and given a DRE, PSA, and interview for signs of prostatitis. Only subjects who are negative for all tests will be accepted into the study. The first 10 men meeting the criteria will comprise the subject group.

Subject Group 1

A small sample of men are given 10 mL polypropylene centrifuge tubes for home-collection of whole ejaculate samples. The tubes are a specific brand that have been verified to release no more than fg amounts of zinc, i.e., amounts below our detection limits, into aqueous solutions. The collection of the ejaculate is done using methods designed to minimize contamination of the fluid with stray zinc and the ejaculate will be stored for not more than 12 hours in refrigerators (˜4° C.) prior to bringing to the laboratory.

Samples from this group are used strictly for developing appropriate methods of sample preparation, separation, fractionation, dissolution and so forth. For the analytical (SIDMS) analyses, these samples are spiked with known amounts of a stable zinc isotope (⁶⁴Zn or ⁶⁶Zn) at specific points along the processing, so that the instrumental analysis can determine the total processing blank, the total processing recovery, and so forth (14).

Subject Group 2

This sample is chosen carefully to represent the population at risk of prostate cancer, for whom cancer screening tests will be most important. Thus, age, PSA levels, DRE status, and family history relating to prostate cancer are all considered in order to assemble an appropriate sample. Subjects include men between 50 and 55 who have (i) PSA levels for at least 2 successive years lower than 2.5 and not rising; (ii) negative findings on a DRE; (iii) no familial disposition towards aggressive prostate cancer; and (iv) no current prostate complaints/dysfunction. The goal of this analysis is to establish the means, variances, and therefore 95% confidence intervals for the zinc concentrations in the various fractions of ejaculate that are analyzed.

Subject Group 3

This group is used for tests for zinc changes in cancer. Thus, this group is comprised of patients who are in the same age range as the controls, but who have recently been diagnosed with prostate cancer.

Analytical Methods for Ejaculate

The stable isotope dilution mass spectrometry (SIDMS) method has been described above. This approach allows one to monitor contamination and loss of analyte throughout the entire process of sample collection, separation, and dissolution so that true absolute values of the analytes can be determined.

Ejaculate is collected in tubes certified to neither remove zinc from samples by absorption or adsorption nor contaminate the samples within the limits of detection. Because semen has about 1000-fold more zinc than any other biological fluid, contamination will be less of a problem than usual in this type of work.

Ejaculate and other samples, e.g., but not limited to, seminal plasma or specific protein fractions, are spiked with a precisely measured amount of ⁶⁴Zn or ⁶⁶Zn before subjected to dissolution procedures to reduce them to elemental composition. The SIDMS sample preparation room is a class-100 clean room within which personnel wear clean-room over-garments and hair covers. All reagents are double-distilled in the laboratory in quartz stills, and made using ultrapure grade materials and 18 MOhm or better grade de-ionized water. Critical sample contact surfaces are all TFE teflon, polypropylene or quartz. For small sample determinations, it has been previously established that the error variance of the whole-process blank for zinc is no greater than ˜2 ng S.D. (14).

Sample preparation after spiking generally progresses by (i) lyophilization; (ii) weighing; (iii) dissolution to elemental composition in concentrated hot nitric acid or perchloric; (iv) purification of zinc by ion exchange; (v) determination of Zn66/64 ratio in the Isotope ratio Mass Spectrometer; and (vi) calculation of initial zinc concentration in the sample.

Fractionation of semen components is performed according to the methods described above. Total zinc content of the fractions can be determined by the apoCA fluorimetric method for free zinc and by the stable isotope dilution mass spectrometry for total zinc. Total seminal plasma zinc and fractional zinc levels will be compared to total levels of seminal plasma prostate specific antigen (PSA) and blood serum PSA determined using commercially available immunoassay (Roche Diagnostics). Free PSA and bound PSA can also be determined using commercially-available tests together with procedures in the literature.

To measure free zinc in material that is not in solution, as for example in the cytosol of individual spermatozoa or in seminal globular proteins, the microspectrofluorimetric methods can be used as described above.

### EXAMPLE 9

Histochemical Imaging of Prostate

These studies address the basic cell biology of zinc in prostate and the commercial goal of imaging the prostate for cancer diagnosis. Results from these studies will give important insights into the fundamentals of zinc metabolism and allow zinc testing of biopsy material to be included as an additional method of diagnosing prostate cancer.

The tissues to be used in this work include prostates harvested from normal men who died without any prostate disease and prostates harvested by prostatectomy or by autopsy from men who had confirmed aggressive prostate cancer. The tissues will be frozen without fixative within an 8-hour postmortem interval. This can include tissues in existing tissue banks, so long as the tissue is frozen without fixative within 0-8 hours postmortem.

Determinations of Tissue Distribution of Total Zinc by Synchrotron-Induced X Ray Fluorescence Imaging

Frozen sections are cut and mounted on glass slides (1 series) and on mylar slides (1 series). The glass-mounted tissue is fixed over aldehyde vapor, then in aldehyde solution for conventional immunostaining to identify various prostate cytoarchitectonic regions. The mylar-mounted sections are sealed in dust-free containers and then sent for processing by synchrotron-induced X Ray fluorescence imaging.

Distribution of Free Zinc at the Macroscopic and Light Microscopic Level

Fresh-frozen tissue sections are stained with either TSQ or Newport Green (cell permeable) or Zinpyr for imaging of the intracellular zinc pools. It is worth noting that the different stains show different “pools” of zinc in the tissue. Thus, for example, the lipophilic stains (TSQ and Zinpyr) will readily stain zinc that is sequestered in the secretory granules or zincosomes in which it is most highly concentrated. Newport green and apoCA-ABDN, on the other hand, will vividly stain cytoplasmic zinc (24) but cannot penetrate these zincosomes and will not stain those cell compartments. Thus, comparison of the differences in staining would indicate subcellular localization of zinc.

Localization of Zinc at the High-Magnification Light and Electron-Microscopic Level

The silver methods of Danscher are the only method of choice. For the silver staining or autometalography (AMG), the tissue is sectioned frozen, then exposed to sulphide vapor (HS) while kept frozen. This treatment precipitates zinc as ZnS in the frozen tissue, thus immobilizing it in situ in whatever subcellular organelles it happens to be. After sulphide precipitation, the tissue is fixed by further exposure to aldehyde vapor (still frozen) before conventionally fixed by aldehyde immersion. Next the tissue sections are developed in a silver developer solution in which the ZnS crystals catalyze reduction of silver, forming silver nanoparticles around the ZnS. Developed sections can then be either counter-stained, cleared, and cover-slipped for light microscope analysis; or dehydrated, embedded in plastic, and ultratomed for analysis in electron microscope.

The following references were cited herein:

1. NCI Fact Sheet 5.29, Jan. 11, 2001, on cis.nci.gov of National Cancer Institute.

2. Zaichick et al., Int. Urol. Nephrol., 28:687-694 (1996).

3. Costello and Franklin, Oncology, 59:269-282 (2000).

4. Brys et al., Biol. Trace Elem. Res., 59:145-152 (1997).

5. McCallum et al., Br. J. Urol., 62:565-570 (1988).

6. Ogunlewe and Osegbe, Cancer 63:1388-1392 (1989).

7. Wong et al., Tumour Biol., 21:328-336 (2000).

8. Wilden and Robinson, Br. J. Urol., 47:295-299 (1975).

9. Chang et al., PNAS, 101(5):1129-1134(2004).

10. Benters et al., Biochem. J., 322:793-9 (1997).

11. Gold and Forber, Investigative Urol., 8:231-238 (1970).

12. Kristiansen et al., Histochem. Cell Biol., 11:125-129 (2001).

13. Danscher, Histochemistry, 71:1-16 (1981).

14. Klitenick et al., Analytical Chemistry 55:921-923 (1983).

15. Frederickson et al., Brain Research, 273:335-339 (1983).

16. Thompson et al., J. Neurosci. Meth., 96:35-45 (2000).

17. Li et al., J. Neuroscience (2001).

18. Brown et al., Fertil Steril., 64:612-622 (1995).

19. Moore, J Polymer Sci., A2:835-843 (1964).

20. Fierke and Thompson, Biometals., 141:205-222 (2001).

21. Huang et al., Biochemistry 35:3439-3446 (1996).

22. Frederickson et al., J. Chem. Neuroanat., 5:521-530 (1992).

23. Gao et al., Analyst, 127:1700-4 (2002).

24. Suh et al., J. Histochem. Cytochem., 47:969-972 (1999).

Any patents or publications mentioned in this specification are indicative of the levels of those skilled in the art to which the invention pertains. Further, these patents and publications are incorporated by reference herein to the same extent as if each individual publication was specifically and individually incorporated by reference.

One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

What is claimed is:

1. A method of screening an individual at risk for prostate cancer, comprising:
collecting a semen sample from said individual;
measuring the level of zinc in said sample;
comparing said zinc level from the at risk individual with zinc levels found in a normal individual; and
correlating a decreased zinc level in the individual compared to the normal level to a risk of developing prostate cancer, thereby screening said individual.

2. The method of claim 1, further comprising:
measuring a level of prostate specific antigen in the at risk individual;
comparing said PSA level from the at risk individual with PSA levels found in a normal individual; and
correlating an increased PSA level in the individual compared to the normal level to a risk of developing prostate cancer.

3. The method of claim 1, wherein said semen sample comprises whole seminal fluid or seminal plasma.

4. The method of claim 3, wherein the zinc comprising said semen sample is measured by stable isotope dilution mass spectrometry or fluorescent spectrometry.

5. The method of claim 4, wherein said fluorescent spectrometry is a ratiometric fluorescence method.

6. The method of claim 5, wherein said ratiometric fluorescence method employs an apoCA and a fluorescent reporter.

7. The method of claim 6, wherein said fluorescent reporter is dansylamide, ABDN,

8. The method of claim 4, wherein said fluorescent spectrometry employs a fluorescent probe.

9. The method of claim 8, wherein said fluorescent probe is Zinpyr, a zin-napthopyr, TSQ, Fluo-zinc, or coumazin.

10. The method of claim 9, wherein said Zinpyr is ZP-1, ZP-4 or ZP-8.

11. The method of claim 9, wherein said Zin-napthopyr is ZPN-1.

12. The method of claim 1, wherein said semen sample is spermatozoa, cytosol of spermatozoa or seminal globulin protein.

13. The method of claim 12, wherein the zinc comprising said semen sample is measured by microspectrofluorimetric methods or silver staining autometalography.

14. The method of claim 13, wherein said microspectrofluorimetric methods employs a cell-impermeable stain or TSQ to stain the zinc comprising said semen sample.

15. The method of claim 14, wherein said cell-impermeable stain is Newport Green, a Zinpyr, or a Zin-naphthopyr.

16. The method of claim 15, wherein said Zinpyr is ZP-1, ZP-4 or ZP-8.

17. The method of claim 15, wherein said Zin-napthopyr is ZPN-1.

18. The method of claim 1, wherein the zinc is free zinc, microligand-bound zinc, protein-bound zinc or zinc carried by spermatozoa.

19. A kit for early diagnosis of prostate cancer in an individual, comprising:
a cassette containing a fluorescent probe;
a specimen container for a semen sample comprising a means permeable to free zinc ions but impermeable to said fluorescent probe, said specimen container insertable into said cassette;
a fluorescence reader comprising means to convert fluorescence values of a fluorescent probe-zinc complex to zinc level values; and
a chart to convert said zinc level values to a status of normal prostate, pre-disposition to prostate cancer or prostate cancer in the individual.

20. The kit of claim 19, further comprising a detergent.

21. The kit of claim 20, wherein said detergent is Triton-X 100.

22. The kit of claim 19, further comprising an antibody immobilized on a substrate, said antibody directed against a zinc-binding ligand in semen.

23. The kit of claim 19, wherein said fluorescent probe is apoCA and a fluorescent reporter, Zinpyr, a zin-napthopyr, TSQ, Fluo-zinc, or coumazin.

24. The kit of claim 23, wherein said fluorescent reporter is dansylamide or ABDN.

25. The kit of claim 23, wherein said Zinpyr is ZP-1, ZP-4 or ZP-8.

26. The kit of claim 23, wherein said Zin-napthopyr is ZPN-1.

27. The kit of claim 19, wherein said means permeable to free zinc ions but not to said fluorescent probe is a dialysis membrane or a molecular sieve.

28. The kit of claim 19, wherein said semen sample is whole seminal fluid.

29. A kit for early diagnosis of prostate cancer in an individual, comprising:
a cassette having an apoCA and a fluorescence reporter contained therein;
a specimen container for a semen sample comprising a membrane permeable to free zinc ions but impermeable to said apoCA and said fluorescent reporter, said specimen container insertable into said cassette;
a fluorescence reader comprising means to convert fluorescence values of a holoCA-fluorescence reporter complex to zinc level values; and
a chart to convert said zinc level values to a status of normal prostate, pre-disposition to prostate cancer or prostate cancer in the individual.

30. The kit of claim 29, further comprising a detergent.

31. The kit of claim 30, wherein said detergent is Triton-X 100.

32. The kit of claim 29, further comprising an antibody immobilized on a substrate, said antibody directed against a zinc-binding ligand in semen.

33. The kit of claim 29, wherein said specimen container is a pouch comprising a dialysis membrane.

34. The kit of claim 29, wherein said fluorescent reporter is dansylamide or ABDN.

35. The kit of claim 29, wherein said semen sample is whole seminal fluid.
